(19) United States
(12) Patent Application Publication
(10) Pub. No.: US 2017/0172687 A1
(43) Pub. Date: Jun. 22, 2017

(54) Printed Strain Gauges for Force Measurement

(71) Applicant: President and Fellows of Harvard College, Cambridge, MA (US)

(72) Inventors: Michael J. Smith, North Reading, MA (US); Frank L. Hammond, III, Atlanta, GA (US); Robert J. Wood, Cambridge, MA (US)

(73) Assignee: President and Fellows of Harvard College, Cambridge, MA (US)

(21) Appl. No.: 15/129,820

(22) PCT Filed: Mar. 27, 2015
(86) PCT No.: PCT/US2015/022943

§ 371 Date: Sep. 27, 2016

Related U.S. Application Data
(60) Provisional application No. 61/971,727, filed on Mar. 28, 2014.
(60) Provisional application No. 62/084,841, filed on Nov. 26, 2014.

(51) Int. Cl. A61B 90/00; A61B 17/28; G01B 7/16; A61B 17/062; G01L 5/00; G01L 1/22

(52) U.S. Cl. CPC A61B 90/06; G01L 5/0028; G01L 1/2287; G01B 7/20; A61B 17/062; A61B 17/28; A61B 2017/00929

(57) ABSTRACT

In a method for printing a strain gauge on an instrument for force measurement, a deposition mask is applied to a surface of an instrument. With the deposition mask on the surface of the instrument, a strain gauge material is deposited on at least one portion of the surface exposed by aperture(s) in the deposition mask. Additionally, electrically conductive material is deposited along pathways that connect with the deposited strain gauge material. In one embodiment, the strain gauge is printed on surgical forceps.

### BACKGROUND

Manual manipulation of small or fragile objects, such as surgical needles, specialized medical devices (stents), and soft biological tissues, requires precise sensing and modulation of interaction forces to prevent unintended damage. Due to the low magnitude of instrument-object interaction forces (5-50 mN) and the length of instruments used during manual manipulation, force and tactile information (regarded by many as essential for precise, dexterous micromanipulation) are very difficult, if not impossible, to perceive without some type of feedback.

Several attempts have been made to incorporate force sensors into surgical tools for feedback, including the use of embedded optical-fiber Bragg grating sensors, soft liquid-embedded tactile sensors, and silicon based strain gauges. These solutions, though effective in research experiments, have proven difficult to implement in practice due to expensive sensor fabrication processes (e.g., precisely machining instruments to install sensors), sensor installation challenges (e.g., mounting compliant sensors to rigid tools), and manufacturing complexity (e.g., process and tooling costs).

### SUMMARY

A method for printing a strain gauge on an instrument and providing feedback for the deflection of the instrument upon which the strain gauge is printed are described herein, where various embodiments of the methods may include some or all of the elements, features, and steps described below.

In a method for printing a strain gauge on an instrument for force measurement, a deposition mask is applied to a surface of an instrument. With the deposition mask on the surface of the instrument, a strain-gauge material that has an electrical resistance that changes as a function of deformation is deposited on at least one portion of the surface exposed by aperture(s) in the deposition mask to form a strain gauge. Additionally, electrically conductive material is deposited along pathways that connect with the deposited strain gauge.

In a method for providing feedback, an instrument is used to perform a manipulation task, wherein a deposited strain gauge is positioned on the instrument surface. The instrument is used to manipulate biological tissue, medical devices, or other objects while the strain gauge is used to measure specific instrument deformation (as a proxy for force) as the instrument manipulates the tissue or medical device.

An instrument with a printed strain gauge for force measurement includes (a) a substrate including an electrically insulating surface; (b) a strain gauge deposited as a layer on the electrically insulating surface; and (c) pathways of electrically conductive material on the electrically insulating surface, wherein the pathways of electrically conductive material are electrically coupled with the deposited strain gauge.

The sensor manufacturing process described herein can leverage chemical vapor deposition (CVD), physical vapor deposition (PVD), and precise laser machining technologies to print metallic strain gauges on the surface of surgical instruments. This additive manufacturing process uses laser-patterned masks and vapor deposited layers of dielectric material and sputtered conductive films to build highly-sensitive strain gauges on surfaces of varying composition and curvature. This process has the advantages of (1) inexpensive fabrication, (2) flexibility of sensor design, and (3) ability to print strain gauges onto pre-existing instruments, eliminating the need for specialized machining.

### BRIEF DESCRIPTION OF THE DRAWINGS

FIG. 1 shows a conceptual illustration of a strain gauge 14 printed on the surface of stainless steel forceps 12 for pinch force feedback.

FIG. 2 shows an illustration process steps for printing a strain gauge 14 starting with a bare surface 20, which is coated with poly(p-xylylene) polymer 22, and masks 24′ and 24″ for strain-gauge and copper deposition

FIG. 3 shows a prototype of a strain gauge 14 and bridge circuit 18 components, including a bridge resistor 30 and a copper circuit layer 32, printed on a poly(p-xylylene) base coating 22 on an aluminum bar 28.

FIG. 4 shows a graph of strain gauge output (filtered and raw voltage along with a polynomial fit) under cantilever loading.

FIG. 5 illustrates a bare surface 20 subjected to mechanical and chemical surface treatments to improve adhesion of poly(p-xylylene) polymer 22.

FIG. 6 illustrates a poly(p-xylylene) coating 22 on the surface 20.

FIG. 7 illustrates a laser-cut mask 24 for sputter deposition on the poly(p-xylxylene) coating 22.

FIG. 8 illustrates a strain gauge 24 deposited through the mask 24 onto the poly(p-xylylene) coating 22.

FIG. 9 illustrates a poly(p-xylylene) protective seal 34 coated over the strain gauge 24.

In the accompanying drawings, like reference characters refer to the same or similar parts throughout the different views; and apostrophes are used to differentiate multiple instances of the same or similar items sharing the same reference numeral. The drawings are not necessarily to scale; instead, emphasis is placed upon illustrating particular principles in the exemplifications discussed below.

### DETAILED DESCRIPTION

The foregoing and other features and advantages of various aspects of the invention(s) will be apparent from the following, more-particular description of various concepts and specific embodiments within the broader bounds of the invention(s). Various aspects of the subject matter introduced above and discussed in greater detail below may be implemented in any of numerous ways, as the subject matter is not limited to any particular manner of implementation. Examples of specific implementations and applications are provided primarily for illustrative purposes.

Unless otherwise defined, used or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially, though not perfectly pure, as practical and imperfect realities may apply; e.g., the potential presence of at least trace impurities (e.g., at less than 1 or 2%) can be understood as being within the scope of the description; likewise, if a particular shape is referenced, the shape is intended to include imperfect variations from ideal shapes, e.g., due to manufacturing tolerances. Percentages or concentrations expressed herein can represent either by weight or by volume.

Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described or shown as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly.

Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms, “includes,” “including,” “comprises” and “comprising,” specify the presence of the stated elements or steps but do not preclude the presence or addition of one or more other elements or steps.

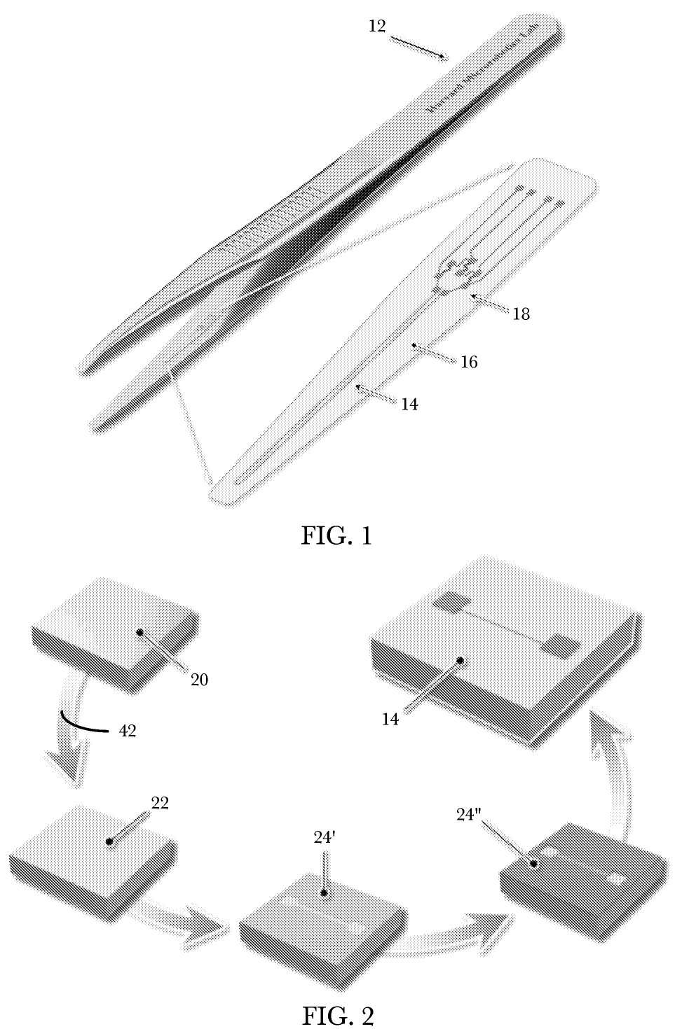

Additionally, the various components identified herein can be provided in an assembled and finished form; or some or all of the components can be packaged together and marketed as a kit with instructions (e.g., in written, video or audio form) for assembly and/or modification by a customer to produce a finished product. As shown in FIG. 1, a strain gauge 14 can be printed onto an instrument, such as commercially available metallic forceps 12, and the ability to accurately measure the tool-tip pinch forces, which cause tool deflection, with this instrument has been demonstrated.

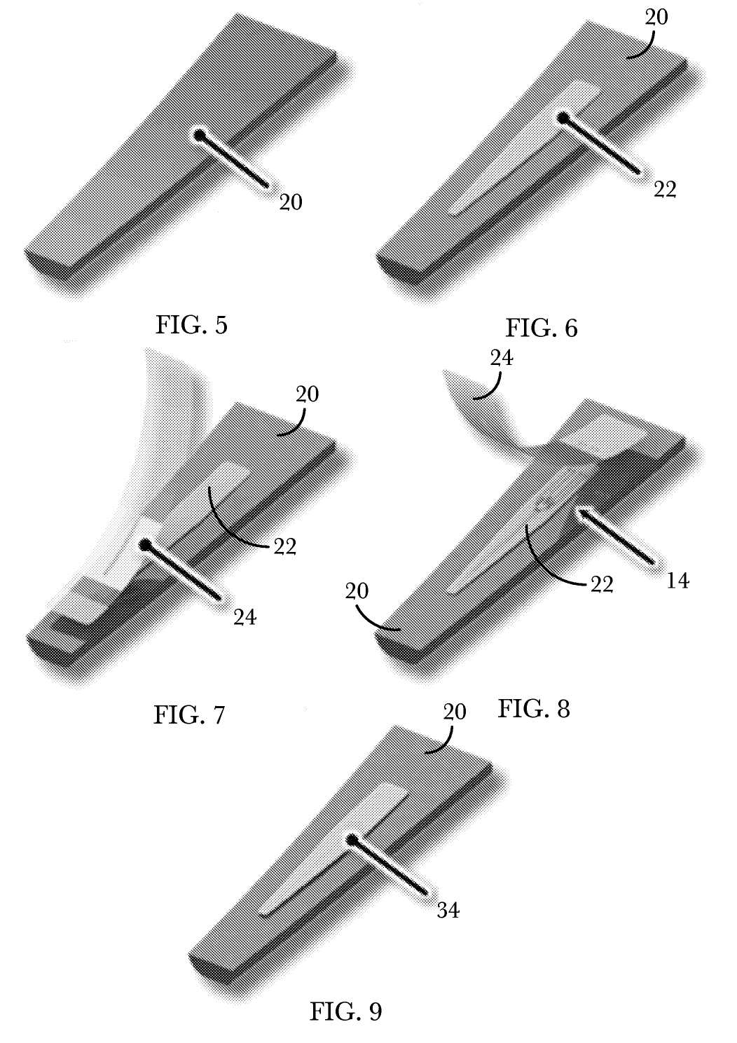

A process for printing strain gauges 14 (as shown in FIGS. 2 and 5-9) involves several steps, including: (1) conditioning of the instrument surface, (2) selective surface masking, (3) deposition of multiple layers of structural and functional materials used to form the sensor components, and (4) a final coating step to protect the sensor. This process is used herein to print strain gauges 14 on the faces of surgical forceps 12 and the surfaces of cantilever bars 28. First, the metallic surfaces of the forceps 12 are sanded using high grit sandpaper to remove any protective surface coatings and to roughen the surfaces. The forceps 12 are then cleaned with acetone to remove any remaining particles and residue, as illustrated in the process schematic shown in FIG. 2. The surfaces are then coated 42 with several-micron-thick layers of PARYLENE C (p-xylylene) polymer, a moisture resistant, low permittivity polymer 22. This coating 22 acts as a substrate layer between deposited metal particles and various surface materials and finishes while electrically insulating the metallic surfaces from the strain gauge 14. PARYLENE coating 42 is performed using a PDS 2010 PARYLENE deposition system (Specialty Coating Systems, Inc., Indianapolis, Ind., USA) to create even coatings on surfaces of varying curvature and size. An additional illustration of fabrication process steps is shown in FIGS. 5-9. In other embodiments, other electrically insulating compositions (e.g., with an electrical resistance at least ten times the electrical resistance of the strain-gauge material) can be used instead of PARYLENE polymer.

After application of a PARYLENE or other insulating coating 22 on a surface 20, as shown in FIG. 6, deposition masks 24 with micron-sized features are laser-cut from a suitable material [e.g., KAPTON polymide tape (DuPont Co., Wilmington, Del., USA)] and tacked onto or positioned upon 44 the surfaces where the strain gauge 14 layers will be deposited. With the deposition masks 24 placed 44 to cover the PARYLENE-coated substrate, as shown in FIGS. 2 and 7, various constituent metal layers, including copper (e.g., for bond pads 36), nichrome and constantan, are sputter deposited onto the surfaces using a physical vapor deposition chamber (Denton Vacuum LLC, Moorestown, N.J., USA). Use of constantan to form the strain gauge 14, shown in FIG. 8, is advantageous because of constantan's high resistivity, versatility, biocompatibility and comparable thermal expansion (15.0 ppm/° C.) with the thermal expansion of 304 stainless steel (17.2 ppm/° C.), of which the rest of the instrument may be formed.

Deposition masks 24 are replaced and/or superimposed over several sputtering cycles to create complex 2.5D conductive elements, circuit electrical traces 32 and contact pads 36, and basic circuit elements, such as resistors 30. After deposition of the strain gauge 14 and its circuit components 18, the forceps' surfaces are coated again with PARYLENE polymer 34 (e.g., a 35-μm-thick coating) for electrical and chemical insulation.

The strain gauge 14 can be coupled via electrically conductive pathways (e.g., having an electrical conductivity that is at least half as high as the conductivity of copper) to a detector that detects changes in electrical resistance through the strain gauge 14 generated by deformation of the strain gauge 14: and the strain (and the force that produces that strain) can then be determined as a function of the resistance change.

Following successful manufacture of the gauge material integrated to the structural substrate and with the provision of the electrically conductive pathways, the strain gauge 14 is connected to a signal conditioning circuit 18 where the circuit output is monitored and/or utilized for force magnitude observation, limit trigger and/or data gathering.

The strain gauge 14 is designed to detect interaction forces encountered by the instrument so that force magnitude can be accurately sensed given gauge calibration data and a linear elastic assumption. In an exemplary microsurgery application, where the grasper interacts with mm-scale nerves and vessels, the gauge 14 is required to sense distal loads up to 1 N with a force resolution orders of magnitude lower (e.g., 20 mN). This requirement places an upper-bound on the noise floor of the sensor after signal conditioning.

Given the design requirements set forth in the previous section, the geometry of the gauge pattern can be designed with several considerations in mind. The foremost design challenge is to maximize the gauge factor, Sₑ, while minimizing the overall footprint, where:

$${S}_{e} = \frac{Δ {R}_{S} / {R}_{S}}{ɛ} , \qquad ( 1 )$$

wherein R_{S} is the nominal gauge resistance, ΔR_{S} is the resistance change induced by mechanical deformation, and e is the material strain. Assuming a linear elastic, isotropic gauge material, for a given gauge configuration, we can express the resistance equation, R=pl/A, as a function of applied strain, e, to obtain an analytical model for the change in resistance assuming uniaxial loading, as follows:

$$Δ {R}_{s} ( ɛ ) = ρ {\sum }_{i = 1}^{N} \frac{{l}_{i}}{{w}_{i} {t}_{i}} ( \frac{( 1 + ɛ )}{[ {( 1 - v ɛ )}^{2} ]} - 1 ) , \qquad ( 2 )$$

where p is the resistivity of the gauge material, and v is the Poisson ratio. Assuming a complex geometry, we have summed resistance contributions from each discrete feature (length, lᵢ; width, wᵢ; and thickness, tᵢ) of the gauge pattern.

From Equation (2), the sensitivity of the gauge 14 is directly proportional to length, lᵢ, and inversely proportional to cross-sectional area, A_{c,I}=wᵢtᵢ.

Experimental validation of the printed strain gauges 14 was conducted using an Instron 5540 Series electromechanical testing system (Instron Inc., Norwood, Mass. USA). The surgical forceps 12 and aluminum bars 28 (cantilevers) fitted with printed nichrome and constantan strain gauges 14 were mounted in a cantilever configuration to examine strain gauge sensitivity and robustness under applied loads. The resistance changes in the strain gauges 14 were measured using a conditioning circuit designed for sensitivity to resistance elements on the order of 100 Ohms-10 kOhm. The forceps 12 and blanks were loaded under several Newtons of force using a set displacement rate of 3 mm/min normal to their surfaces.

In addition to mechanical characterization, the thermal characteristics of the gauge 14 can be adequately determined to quantify stability of the sensing system when operating in environments with varying temperatures. The thermal expansion of the steel structural material induces strain in the gauge material, resulting in resistance drifts as a function of temperature. The change in resistance due to temperature gradients [ΔR_{S}(ΔT)] can be computed via the following equation:

$$Δ {R}_{s} ( Δ T ) = ρ {\sum }_{i = 1}^{N} \frac{{l}_{i}}{{w}_{i} {t}_{i}} ( \frac{( 1 + α Δ T )}{[ {( 1 - v α Δ T )}^{2} ]} - 1 ) . \qquad ( 3 )$$

### Results:

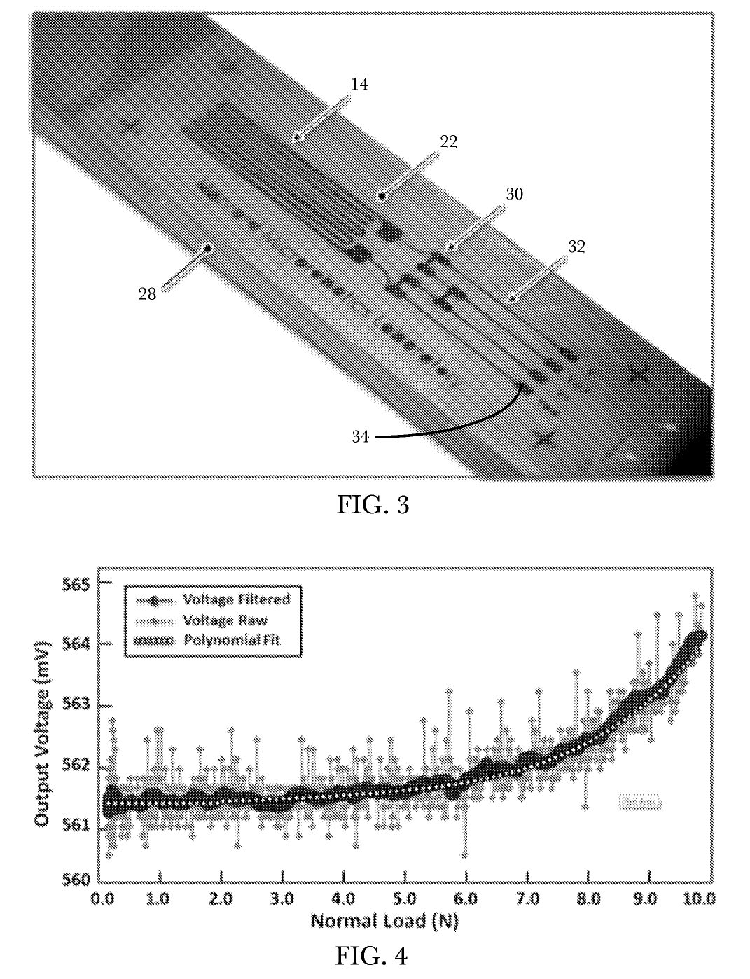

The printed strain gauge fabrication process proved robust against variations in instrument surface shape, roughness, and material, as demonstrated in strain gauge and circuit printing on a flat aluminum blank (as shown in FIG. 3) and on a roughened stainless steel forceps surface. The robustness of the fabrication process to surface complexity seems limited primarily by deposition mask compliance and the concavity of the target sputtering surfaces (occlusion of line-of-sight). In preliminary electromechanical tests, a constantan strain gauge 14 (in the form of a 200 μm×1.0 cm×300 nm trace) printed onto a cantilever surface exhibited sensitivity to forces between 0-10N, a scale appropriate for sensing expected manipulation forces, as shown in FIG. 4). A polynomial fit to the force-output curve shows a 0.0023 V/N average sensitivity without amplification (assuming small deformation).

The response of the constantan gauge 14 was non-monotonic. Sudden changes in output voltage as normal force increased were likely due to both the propagation of cracks in the constantan gauge 14 as it reaches its strain limits and in part to friction between test specimens and the testing device. After significant deformation, the sensitivity and range of the strain gauge 14 permanently degrades, with the base gauge resistance returning to increasingly higher values as cracks continue to form, eventually rendering the gauge 14 useless.

### Interpretation:

Initial validation experiments demonstrate that printing strain gauges 14 on surgical instruments for force measurement is feasible, while leaving room for improvement in the fabrication process and sensor designs to increase strain gauge mechanical robustness, signal to noise ratio (SNR), lower ranges of sensitivity (μNs), and process yield and repeatability. Mechanical robustness of the printed strain gauges 14 can be increased by replacing nichrome with constantan, which has a higher strain capacity and can sustain sensitivity under large deformations. Lower sensitivity ranges and better SNR can be achieved by including signal conditioning elements in the gauge circuit 18 and increasing the length of gauge 14 along the axis of the instrument surface where the most deformation occurs. Process yield can be improved by refining the mask alignment and application process (using alignment marks) to ensure more-precise dielectric and metal deposition patterns and by designing more assembly-focused sensor layups.

### Deposition on a Multi-Layer Laminate Structure:

In various embodiments, the strain gauge 14, described herein, can be deposited on a pop-up, multi-layer laminated structure, as described in published PCT Application No. WO 2012/109559 A1 and in US Provisional Patent Application No. 61/862,066, filed on 4 Aug. 2013. As described in these earlier applications, the layers in the laminate structure can include at least one rigid layer and at least one flexible layer, wherein the rigid layer includes a plurality of rigid segments, and the flexible layer can extend between the rigid segments to serve as a joint. The flexible layers are substantially less rigid than the rigid layers, wherein the rigid layer can have a rigidity that is at least twice as great as or an order of magnitude greater than (e.g., greater than 10× or greater than 100×) the rigidity of the flexible layer; likewise, the flexible layer can have at least 10 times or at least 100 times the flexibility of the rigid layers. The layers can then be stacked and bonded at selected locations to form a laminate structure with inter-layer bonds, and the laminate structure can be distorted or flexed to produce an expanded three-dimensional structure, wherein the layers are joined at the selected bonding locations and separated at other locations. Support circuitry 18 can likewise be deposited along with the strain gauge 14 on the top surface of a multi-layer laminate structure. In one embodiment, the multi-layer laminate structure onto which the strain gauge 14 is deposited can be a micro-surgical grasper, as described in US Provisional Patent Application No. 61/862,066, formed, e.g., of layers of 304 stainless steel, KAPTON polyimide, and acrylic adhesive. Specifically, the strain gauge 14 can be deposited on an outer surface of a jaw of the grasper.

### Additional Applications:

In additional applications, strain gauges 14 can be deposited on a variety of equipment where measuring strain may be helpful. For example, strain gauges 14 can be deposited via these methods on sports equipment (such as baseball bats, golf clubs, punching bags, etc.) without compromising/significantly effecting the structure and performance of the equipment, yet enabling strain measurements that can be correlated with force outputs from the athlete as means to track athlete performance levels.

In describing embodiments of the invention, specific terminology is used for the sake of clarity. For the purpose of description, specific terms are intended to at least include technical and functional equivalents that operate in a similar manner to accomplish a similar result. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties or other values are specified herein for embodiments of the invention, those parameters or values can be adjusted up or down by 1/100^{th}, 1/50^{th}, 1/20^{th}, 1/10^{th}, ⅕^{th}, ⅓^{rd}, ½, ⅔^{rd}, ¾^{th}, ⅘^{th}, 9/10^{th}, 19/20^{th}, 49/50^{th}, 99/100^{th}, etc. (or up by a factor of 1, 2, 3, 4, 5, 6, 8, 10, 20, 50, 100, etc.), or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention. Further still, other aspects, functions and advantages are also within the scope of the invention; and all embodiments of the invention need not necessarily achieve all of the advantages or possess all of the characteristics described above. Additionally, steps, elements and features discussed herein in connection with one embodiment can likewise be used in conjunction with other embodiments. The contents of references, including reference texts, journal articles, patents, patent applications, etc., cited throughout the text are hereby incorporated by reference in their entirety; and appropriate components, steps, and characterizations from these references may or may not be included in embodiments of this invention. Still further, the components and steps identified in the Background section are integral to this disclosure and can be used in conjunction with or substituted for components and steps described elsewhere in the disclosure within the scope of the invention. In method claims, where stages are recited in a particular order—with or without sequenced prefacing characters added for ease of reference—the stages are not to be interpreted as being temporally limited to the order in which they are recited unless otherwise specified or implied by the terms and phrasing.

What is claimed is:

1. A method for printing a strain gauge on an instrument for force measurement, the method comprising:
applying a mask to a surface of an instrument, wherein the mask defines at least one aperture;
with the mask on the surface of the instrument, depositing a strain-gauge material that has an electrical resistance that changes as a function of deformation on at least one portion of the surface exposed by the aperture(s) of the mask to form a strain gauge; and
depositing electrically conductive material along pathways that connect with the deposited strain gauge.

2. The method of claim 1, further comprising, before depositing the strain-gauge material, depositing an electrically insulating composition on the instrument surface.

3. The method of claim 2, wherein the electrically insulating composition comprises a p-xylylene polymer.

4. The method of claim 2, further comprising, before depositing the electrically insulating composition:
cleaning the surface to remove any remaining particles and residue; and then
preparing the surface via at least one of mechanical and chemical preparation to promote adhesion and remove any protective coatings.

5. The method of claim 1, wherein the strain-gauge material comprises constantan.

6. The method of claim 1, wherein the deposited electrically conductive material forms at least one of an electrical trace and a contact pad.

7. The method of claim 6, wherein the electrically conductive material comprises copper.

8. The method of claim 6, further comprising depositing a resistor along the pathway of the electrically conductive material.

9. The method of claim 1, further comprising depositing a protective polymer coating on the deposited strain gauge.

10. The method of claim 9, wherein the protective polymer comprises a p-xylylene polymer.

11. The method of claim 1, wherein the instrument is selected from forceps and a surgical needle.

12. The method of claim 1, wherein the instrument is a piece of sports equipment.

13. The method of claim 1, wherein the instrument comprises a cantilever bar.

14. The method of claim 1, wherein the strain gauge has dimensions no greater than about 1 cm.

15. A method for providing instrument deformation feedback, comprising:
manipulating tissue or a medical device with an instrument upon which a strain gauge is deposited; and
measuring strain with the strain gauge as the instrument manipulates the tissue or medical device.

16. The method of claim 15, wherein strain is measured by detecting resistance changes through the strain gage using a signal-conditioning circuit.

17. The method of claim 16, wherein the measured change in resistance is correlated with the applied force that produces the strain.

18. An instrument with a printed strain gauge for force measurement, the instrument comprising:
a substrate including an electrically insulating surface;
a strain gauge deposited as a layer on the electrically insulating surface; and
pathways of electrically conductive material on the electrically insulating surface, wherein the pathways of electrically conductive material are electrically coupled with the deposited strain gauge.

19. The instrument of claim 18, wherein the instrument is selected from forceps and a surgical needle.
